(19) United States
(12) Patent Application Publication
(10) Pub. No.: US 2024/0404641 A1
(43) Pub. Date: Dec. 5, 2024

(54) GRAPH-BASED ARTIFICIAL INTELLIGENCE (AI) ALIGNMENT OF FUNCTIONAL ANNOTATIONS FROM DIFFERENT ONTOLOGIES USING BIOLOGICAL SEQUENCES

(71) Applicant: INTERNATIONAL BUSINESS MACHINES CORPORATION, Armonk, NY (US)

(72) Inventors: Gowri NAYAR, San Jose, CA (US); Harsha Vardhan KRISHNAREDDY, Sunnyvale, CA (US); Laura-Jayne GARDINER, Wirral (GB); Ritesh Vijay KRISHNA, Sale (GB)

(21) Appl. No.: 18/327,473

(22) Filed: Jun. 1, 2023

(51) Int. Cl. G16B 50/10; G16B 30/10; G16B 40/20

(52) U.S. Cl. CPC G16B 50/10; G16B 30/10; G16B 40/20

(57) ABSTRACT

Aspects of the present disclosure relate generally to bioinformatics and, more particularly, to systems, computer program products, and methods of mapping functional annotations of genes and proteins from different ontologies. For example, a computer-implemented method includes: identifying, by a processor set, associations of nodes between a first graph of a gene ontology capturing biological processes and a second graph of a protein function ontology; generating, by the processor set, a composite graph by merging the first graph and the second graph using the associations of nodes; adding, by the processor set, node embeddings for nodes of the composite graph; determining, by the processor set, at least one new association of unassociated nodes of the composite graph using the node embeddings; and saving at least one new association of unassociated nodes of the composite graph as an association of nodes between the first graph and the second graph in persistent storage.

### BACKGROUND

Aspects of the present invention relate generally to bioinformatics and, more particularly, to systems, computer program products, and methods of mapping functional annotations of genes and proteins from different ontologies.

Gene ontologies provide a systematic representation of knowledge about genes that supports a more precise classification of genes and the relationships among genes in the classification. For instance, the Gene Ontology (GO) describes gene products with three independent categories: biological process, cellular component, and molecular function. Scientists and researchers author functional annotations that report connections between gene products such as proteins and the biological types represented in the Gene Ontology. Other ontologies, such as the Kyoto Encyclopedia of Genes and Genomes (KEGG), group genes into pathways that list genes participating in the same biological process. Still other ontologies provide information about gene products such as InterPro that provides functional analysis of proteins and classifies proteins into families. These different ontologies provide different representations of knowledge about genes and gene products. Scientist and researchers in the bioinformatics community typically consult these different ontologies to understand and derive biological insight from genome data in their work.

### SUMMARY

In a first aspect of the invention, there is a computer-implemented method including: identifying, by a processor set, associations of nodes between a first graph of a gene ontology capturing biological processes and a second graph of a protein function ontology; generating, by the processor set, a composite graph by merging the first graph and the second graph using the associations of nodes; adding, by the processor set, node embeddings for nodes of the composite graph; determining, by the processor set, at least one new association of unassociated nodes of the composite graph using the node embeddings; and saving, by the processor set, the at least one new association of unassociated nodes of the composite graph as an association of nodes between the first graph and the second graph in persistent storage.

In another aspect of the invention, there is a computer program product including one or more computer readable storage media having program instructions collectively stored on the one or more computer readable storage media. The program instructions are executable to: identify associations of nodes between a first graph of a gene ontology capturing biological processes and a second graph of a protein function ontology; generate a composite graph by merging the first graph and the second graph using the associations of nodes; perform node embedding including node features of biological sequences for nodes of the composite graph; determine at least one new association of unassociated nodes of the composite graph using the node embeddings; and save the at least one new association of unassociated nodes of the composite graph as an association of nodes between the first graph and the second graph in persistent storage.

In another aspect of the invention, there is a system including a processor set, one or more computer readable storage media, and program instructions collectively stored on the one or more computer readable storage media. The program instructions are executable to: identify associations of nodes between a first graph of a gene ontology capturing biological processes and a second graph of a protein function ontology; generate a composite graph by merging the first graph and the second graph using the associations of nodes; perform node embedding for nodes of the composite graph; build a graph neural network from the composite graph with the node embeddings; perform link prediction using the graph neural network to identify at least one new association of unassociated nodes of the composite graph; and save the at least one new association of unassociated nodes of the composite graph as an association of nodes between the first graph and the second graph in persistent storage.

### BRIEF DESCRIPTION OF THE DRAWINGS

Aspects of the present invention are described in the detailed description which follows, in reference to the noted plurality of drawings by way of non-limiting examples of exemplary embodiments of the present invention.

FIG. 1 depicts a computing environment according to an embodiment of the present invention.

FIG. 2 shows a block diagram of an exemplary environment in accordance with aspects of the present invention.

FIG. 3 depicts an illustration of an exemplary diagram of a workflow in accordance with aspects of the present invention.

FIG. 4 shows a flowchart of an exemplary method in accordance with aspects of the present invention.

FIG. 5 shows a flowchart of an exemplary method in accordance with aspects of the present invention.

### DETAILED DESCRIPTION

Aspects of the present invention relate generally to bioinformatics and, more particularly, to systems, computer program products, and methods of mapping functional annotations of genes and proteins from different ontologies. More specifically, aspects of the present invention relate to methods, computer program products, and systems for generating a first graph based on information from a gene ontology capturing biological processes, generating a second graph based on information from a protein function ontology, identifying associations of nodes between the first graph of the gene ontology capturing biological processes and the second graph of the protein function ontology, generating a composite graph by merging the first graph and the second graph using the associations of nodes, and determining new associations of unassociated nodes of the composite graph using node embeddings added to nodes of the composite graph and graph-based artificial intelligence (AI) that automatically and computationally infers mappings between terms of the gene ontology capturing biological processes and the protein function ontology. Functional annotations available in these ontologies were manually curated over decades of effort. Unfortunately, manually curated mappings between terms of gene and protein ontologies with functional annotations has been limited to a small subset of terms in these ontologies and, consequently, queries for protein sequences, for instance, can result in partial or no functional annotations available when querying these resources individually. According to aspects of the present invention, the methods, systems, and computer program products described herein automatically and computationally infer mappings between terms of gene and protein ontologies and can provide functional annotations available in these ontologies for queries.

In embodiments, the methods, systems, and computer program products described herein graph the terms and the hierarchical relationship between terms of a gene ontology, likewise graph the terms and the hierarchical relationship between terms of a protein ontology, and generate a composite graph by merging the graphs of the gene and the protein ontologies using associations of the terms of these ontologies. The methods, systems, and computer program products of the present disclosure perform node embedding, including node features of biological sequences, for nodes of the composite graph and computationally infer new node associations for unassociated nodes of the composite graph that map terms of the gene and protein ontologies in embodiments. A graph neural network is generated in embodiments from the composite graph with the node embeddings, and the methods, systems, and computer program products described herein perform link prediction to find new node associations of the graph neural network.

Aspects of the present invention are directed to improvements in computer-related technology and existing technological processes in bioinformatics for mappings terms of gene and protein ontologies and providing functional annotations available in these ontologies for queries, among other features as described herein. In embodiments, the methods, computer program products, and systems may generate a first graph based on information from a gene ontology capturing biological processes, generate a second graph based on information from a protein function ontology, identify associations of nodes between the first graph of the gene ontology capturing biological processes and the second graph of the protein function ontology, generate a composite graph by merging the first graph and the second graph using the associations of nodes, and determine new associations of unassociated nodes of the composite graph using node embeddings added to nodes of the composite graph and graph-based artificial intelligence (AI) that automatically and computationally infers mappings between terms of the gene ontology and the protein ontology. Advantageously, the methods, computer program products, and systems described herein automatically and computationally infer mappings between terms of gene and protein ontologies and provide functional annotations available in these ontologies for queries. These are specific improvements in existing technological processes in bioinformatics for mappings terms of gene and protein ontologies and providing functional annotations available in these ontologies for queries.

Implementations of the disclosure describe additional elements that are specific improvements in the way computers may operate and these additional elements provide non-abstract improvements to computer functionality and capabilities. As an example, the methods, computer program products, and systems describe graph build module, term mapping module, node embedding module, graph merging module, node ranking module, multiple sequence alignment (MSA) module, feature module, graph neural network (GNN) build module, GNN module, and graph neural network that generate a first graph based on information from a gene ontology capturing biological processes, generate a second graph based on information from a protein function ontology, identify associations of nodes between the first graph of the gene ontology capturing biological processes and the second graph of the protein function ontology, generate a composite graph by merging the first graph and the second graph using the associations of nodes, and determine new associations of unassociated nodes of the composite graph using node embeddings added to nodes of the composite graph and graph-based artificial intelligence (AI) that automatically and computationally infers mappings between terms of the gene ontology and the protein ontology. The additional elements of the methods, computer program products, and systems of the present disclosure are specific improvements in the way computers may operate to automatically and computationally map terms of gene and protein ontologies and provide functional annotations available in these ontologies for queries.

It should be understood that, to the extent implementations of the invention collect, store, or employ personal information provided by, or obtained from, individuals, such information shall be used in accordance with all applicable laws concerning protection of personal information. Additionally, the collection, storage, and use of such information may be subject to consent of the individual to such activity, for example, through “opt-in” or “opt-out” processes as may be appropriate for the situation and type of information. Storage and use of personal information may be in an appropriately secure manner reflective of the type of information, for example, through various encryption and anonymization techniques for particularly sensitive information.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as graph-based AI alignment of functional annotations of ontologies code of block 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

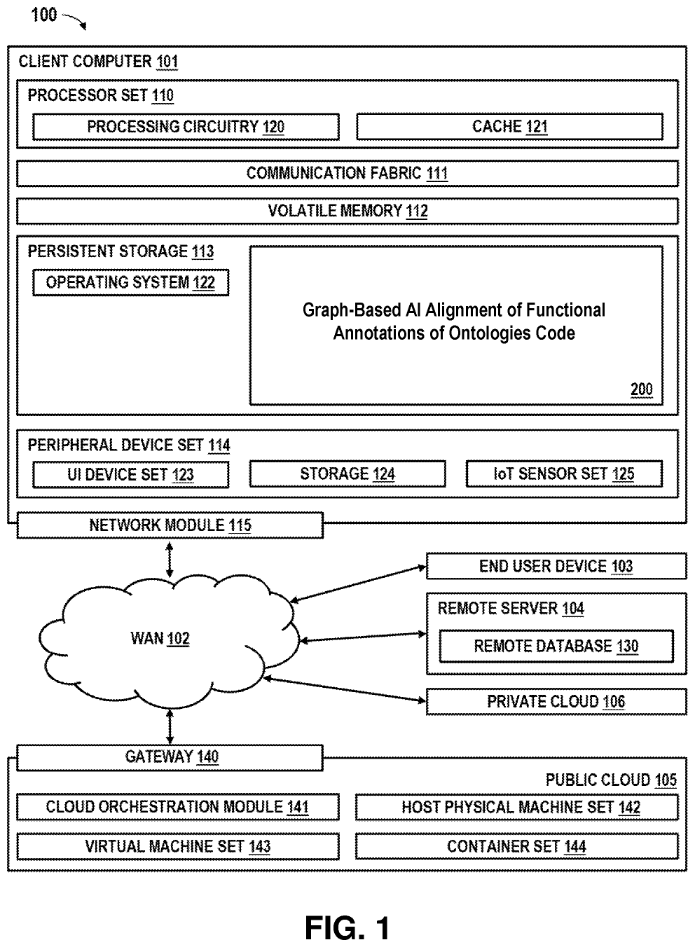

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economics of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

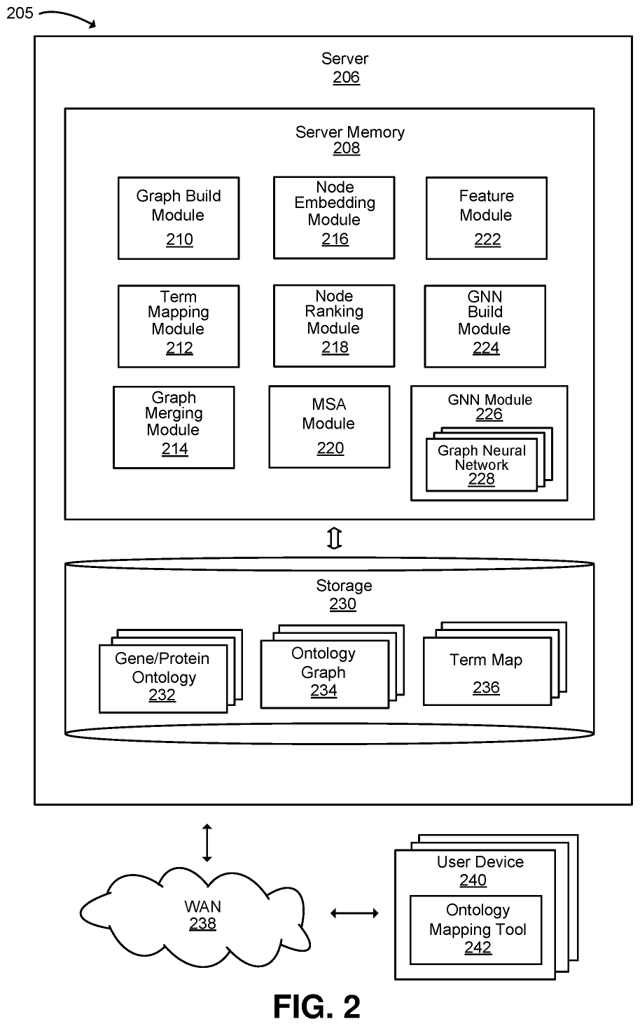

FIG. 2 shows a block diagram of an exemplary environment 205 in accordance with aspects of the present invention. In embodiments, the environment includes a server 206, which may be a computer system such as a computer 101 described with respect to FIG. 1 with which remote servers 104 and public cloud 105, each also described with respect to FIG. 1, may communicate over a network such as WAN 102 described with respect to FIG. 1. In general, server 206 supports services for mapping functional annotations of genes and proteins from different ontologies.

Server 206 has server memory 208 such as volatile memory 112 described with respect to FIG. 1. Server 206 includes, in memory 208, graph build module 210 having functionality to receive terms of an ontology and their hierarchical relationships from an ontology, derive a directed acyclic graph with vertices representing the terms of the ontology and edges representing the hierarchical relationship between terms of the ontology, and save the graph in embodiments. Server 206 includes, in memory 208, term mapping module 212 having functionality to map vertices or nodes with corresponding terms of ontologies between directed acyclic graphs and save the mapping in embodiments. Server 206 includes, in memory 208, graph merging module 214 having functionality to derive a composite directed acyclic graph from two different acyclic graphs by merging the two different graphs with known associations.

Server 206 also includes, in memory 208, node embedding module 216 having functionality to embed nodes of the composite graph with node attributes such as features from biological sequences in embodiments. Server 206 includes, in memory 208, node ranking module 218 having functionality to rank associations of nodes using node embeddings in embodiments. Server 206 includes, in memory 208, MSA module 220 having functionality to perform multiple sequence alignment to identify representative biological sequences for nodes of the composite graph in embodiments. Server 206 includes, in memory 208, feature module 222 having functionality to select representative biological sequences as features for nodes of the composite graph and assigning the features as node attributes to nodes of the composite graph in embodiments.

Server 206 further includes, in memory 208, GNN build module 224 having functionality to build a graph neural network (GNN) from the composite graph with node embeddings including features of biological sequences as node attributes in embodiments. Server 206 further includes, in memory 208, GNN module 226 having functionality to perform link prediction to find node associations of the graph neural network 228. GNN module 226 may include graph neural network 228 built by GNN build module 224. In general, the composite graph with the embedded nodes is input to build the GNN and the embedded nodes are mapped to a d-dimensional embedding space and passed through a series of neural networks. The output of the GNN is a matrix of node embeddings with weighted probabilities from the information provided by the series of neural networks. The GNN may be trained using the composite graph and known node associations to perform link prediction to predict a connection between nodes for a given input term based on the matrix of probabilities from information extracted from node embeddings output by the GNN. Accordingly, the trained graph neural network can predict a protein that maps to a given gene and can predict a gene that maps to a given protein.

In embodiments, the graph build module 210, term mapping module 212, graph merging module 214, node embedding module 216, node ranking module 218, MSA module 220, feature module 222, GNN build module 224, and GNN module 226 may each comprise modules of the code of block 200 of FIG. 1. Such modules may include routines, programs, objects, components, logic, data structures, and so on that perform particular tasks or implement particular data types that the code of block 200 uses to carry out the functions and/or methodologies of embodiments of the invention as described herein. These modules of the code of block 200 are executable by the processing circuitry 120 of FIG. 1 to perform the inventive methods as described herein. The server 206 may include additional or fewer modules than those shown in FIG. 2. In embodiments, separate modules may be integrated into a single module. Additionally, or alternatively, a single module may be implemented as multiple modules. Moreover, the quantity of devices and/or networks in the environment is not limited to what is shown in FIG. 2. In practice, the environment may include additional devices and/or networks; fewer devices and/or networks; different devices and/or networks; or differently arranged devices and/or networks than illustrated in FIG. 2.

In accordance with aspects of the present invention, server 206 of FIG. 2 also includes storage 230 which may be storage such as storage 124 of computer 101 described with respect to FIG. 1. Storage 230 may store in files gene/protein ontologies 232 which may be a gene ontology, a protein ontology, or biological sequence ontology. Storage 230 may also store in files ontology graphs 234. The ontology graphs may be a graph of a gene ontology, a protein ontology, and/or biological sequence ontology. Storage 230 may also store in files term map 236 which may include node associations of terms and/or functional annotations of ontology graphs.

In accordance with aspects of the present invention, environment 205 of FIG. 2 also shows user device 240 which may be a computer system such as end user device 103, described with respect to FIG. 1, that may communicate over WAN 238 which may be a wide area network such as WAN 102, described with respect to FIG. 1. User device 240 may include ontology mapping tool 242 that provides an application to query gene and/or protein information in gene protein ontologies 232 and receive information about the gene and/or protein including functional annotations mapped from the gene/protein ontologies 232 using term map 236.

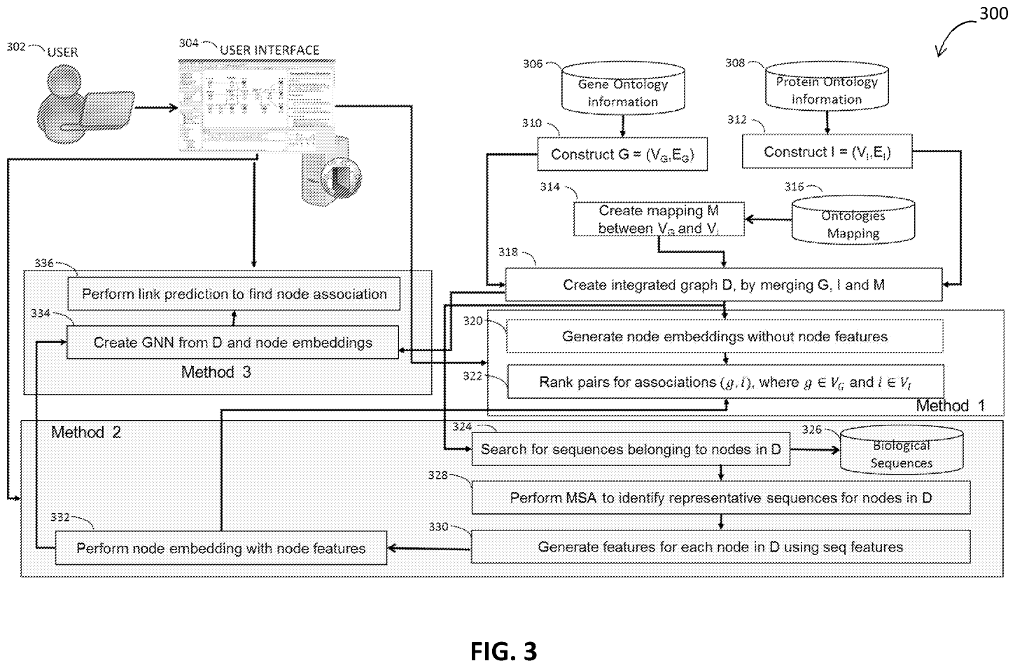

FIG. 3 depicts an illustration of an exemplary diagram of a workflow for mapping functional annotations of genes and proteins from different ontologies in accordance with aspects of the present invention. In general, diagram 300 of FIG. 3 illustrates the workflow of constructing a mapping of functional annotations of genes from a database of gene ontology information 306 and a database of protein ontology information 308 that a user 302 may query by entering information about a gene or protein into user interface 304 of a mapping tool to receive functional annotations from ontologies related to the gene or protein. In particular, diagram 300 of FIG. 3 illustrates workflows of three (3) methods for constructing the mapping of functional annotations of genes from the database of gene ontology information 306 and the database of protein ontology information 308. The workflow of the first method begins by constructing exemplary directed acyclic graph G from the gene ontology information 306 at reference numeral 310, where G=(V_{G},E_{G}), V_{G} is the set of vertices or nodes representing the terms of the ontology, and E_{G} is the set of edges representing the hierarchical relationship between terms of the ontology. At 312, exemplary directed acyclic graph I is constructed from the protein ontology information 308, where I=(V_{I},E_{I}), V_{I} is the set of vertices or nodes representing the terms of the ontology, and E_{I} is the set of edges representing the hierarchical relationship between terms of the ontology. Known relationships between terms of the gene ontology and terms of the protein ontology are retrieved from mapping information of the ontologies in mapping database 316 in embodiments, and a mapping. M, is created at 314 between V_{G} and V_{I} such that the terms of the vertices or nodes of V_{G} from the gene ontology that correspond with the terms of the vertices or nodes of V_{I} from the protein ontology are mapped between graphs G and I. At 318, a composite graph D is generated by integrating graphs G and I using mapping M. For instance, graph N may be constructed as an intermediate graph such that the nodes in N are {V_{G}. V_{I}} and the edges are {E_{G},E_{I}, E_{M}}, where E_{M} are the new edges formed according to the information in mapping, M. The largest connected component (LCC) may be extracted from graph N to form graph D such that D=(V,E) where V and E are the nodes and edges remaining in the LCC of N. At step 320, node embeddings without node features are generated. For example, the node embeddings may be low-dimensional vectors that represent the graph nodes and edges in a vectorial space. To do so, those skilled in the art should appreciate that latent multi-dimensional embedding may be generated for each node in D by applying shallow network embedding techniques that preserve the network neighborhood of nodes of the graph, for example, like the DeepWalk technique and node2vec technique that apply a skip-gram model on generated random walks of the graph.

At step 322, pairs of associations (g,i), where g∈V_{G} and i∈V_{I}, are ranked. For example, where a mapping between given pairs of node associations (g,i) does not exist in M, the proximity of the pair of nodes may be determined in the latent space by exploiting the node embeddings to compute distance measures between the nodes. Two nodes are determined to be close if the distance measure is within a statistical threshold, and, accordingly, the terms of the nodes, each from a respective gene ontology and a protein ontology, are candidates in the ranking results for identifying corresponding terms. In this way, the proximity of the pair of nodes may be determined in the latent space for each node, g, from V_{G} with respect to a given node, i, from V_{I}, where a mapping between given pairs of node associations (g,i) does not exist in M. In embodiments, various strategies can be applied to estimate the statistical threshold by leveraging the information captured through the known relationships in M. For instance, parameters of the distribution generated may be inferred by the distance measures observed between pairs in M and/or by determining the distribution of the length of shortest path between the pairs. A scoring function can be computed by combining certain thresholds to perform a combined ranking. Ranked results may be sent to user interface 304 for display to user 302 for queries entered for information about a gene or protein. In embodiments, the highest ranked pair of node associations (g,i) may be saved in mapping, M, in mapping database 316. The steps of the workflow of the first method are finished at reference numeral 322.

The workflow of the second method uses steps at reference numeral 310 of constructing exemplary directed acyclic graph G. 312 of constructing exemplary directed acyclic graph I. 314 of creating mapping, M, and 318 of generating composite graph D. The steps of the workflow of the second method continue at reference numeral 324 of searching for biological sequences belonging to nodes in D. For example, resources such as databases with sequence ontologies and/or associations of biological sequences with terms of the gene ontology and/or terms of the protein ontology may be mined to collect DNA sequences associated with the respective terms of the ontologies. (Such as the IBM Functional Genomics Platform that is a relational database linking genotype to phenotype for over 300 million biological sequences extracted from microbial genomes.)

At 328, multiple sequence alignment (MSA) may be performed to identify and select representative sequences for nodes in D. Identical sequences present in multiple nodes may be removed so that representative sequences are unique to their respective nodes. In embodiments, there may be a minimum threshold of one (1) and a predetermined maximum threshold number of representative sequences per node, for example two (2) or three (3). Nodes may be removed from D that have no representative sequence. At 330, features of the representative DNA sequences are generated for each node in D. For instance, latent representations may be generated for each node using DNA sequences as features through standard NLP techniques such as seq2seq and word2vec. For example, seq2seq or a sequence-to-sequence technique encodes each DNA sequence as a latent representation such as a context vector. The latent representations output by the encoder are assigned as features to each node in D as node attributes. At 332, node embedding is performed with node features. For example, the node embeddings may include low-dimensional vectors that represent the graph nodes and edges in a vectorial space and context vectors that are latent representations of DNA sequences.

Continuing with the steps of the workflow of the second method illustrated in FIG. 3, pairs of associations (g,i), where g∈V_{G} and i∈V_{I}, are ranked as described above at step 322. For example, where a mapping between given pairs of node associations (g,i) does not exist in M, the proximity of the pair of nodes may be determined in the latent space by exploiting the node embeddings with latent representations of DNA sequences. Two nodes are determined to be close if the distance measure is within a statistical threshold, and, accordingly, the terms of the nodes, each from a respective gene ontology and a protein ontology, are candidates in the ranking results for identifying corresponding terms. In this way, the proximity of the pair of nodes may be determined in the latent space for each node, g, from V_{G} with respect to a given node, i, from V_{I}, where a mapping between given pairs of node associations (g,i) does not exist in M. In embodiments, various strategies can be applied to estimate the statistical threshold by leveraging the information captured through the known relationships in M as described above at step 322. Ranked results may be sent to user interface 304 for display to user 302 for queries entered for information about a gene or protein. In embodiments, the highest ranked pair of node associations (g,i) may be saved in mapping, M, in mapping database 316. The steps of the workflow of the second method are finished upon performing the step of ranking pairs of associations (g,i), where g∈V_{G} and i∈V_{I}, as described above at step 322, and saving the highest ranked pair of node associations (g,i) in mapping, M, in mapping database 316.

The workflow of the third method uses steps at reference numeral 310 of constructing exemplary directed acyclic graph G, 312 of constructing exemplary directed acyclic graph I, 314 of creating mapping, M, 318 of generating composite graph D, 324 of searching for biological sequences, 328 of performing multiple sequence alignment (MSA), 330 of generating features of representative DNA sequences, and 332 of performing node embedding with node features. The steps of the workflow of the third method continue at reference numeral 334 of creating a graph neural network from graph D and node embeddings of D. To do so, graph D and the associated node specific features are translated to train a graph neural network. For example, graph D may be translated to the adjacency matrix of graph D and the associated node specific features are translated to a node attribute matrix. The adjacency matrix and node attribute matrix are input into stacked layers of the graph neural network that perform computational operations using a convolutional operator, a recurrent operator, a sampling operator, and a skip connection operator to propagate information in each layer and a pooling operator that extract high-level information in each layer. The output layer of the graph neural network generates a matrix of probabilities that two nodes of the composite graph are associated from the information in the node embeddings provided by the stacked layers that are trained according to a loss function. The trained graph neural network can predict a protein that maps to a given gene and can predict a gene that maps to a given protein. At step 336, link prediction is performed using the graph neural network to find a node association. In link prediction, the GNN predicts probabilities of connections between nodes based on information extracted from node embeddings output by the GNN. The term of the node predicted to be associated with the term of the node input in a query may be sent to user interface 304 for display to user 302 for queries entered for information about a gene or protein. In embodiments, the terms of the nodes predicted to be associated may be saved in mapping, M, in mapping database 316. The steps of the workflow of the third method are finished upon performing the step of reference numeral 336.

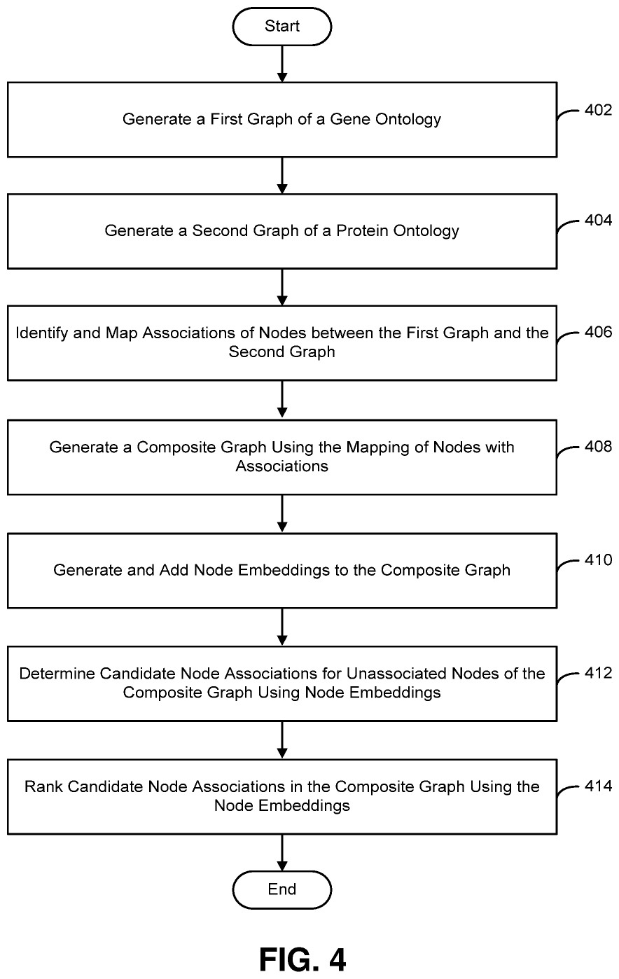

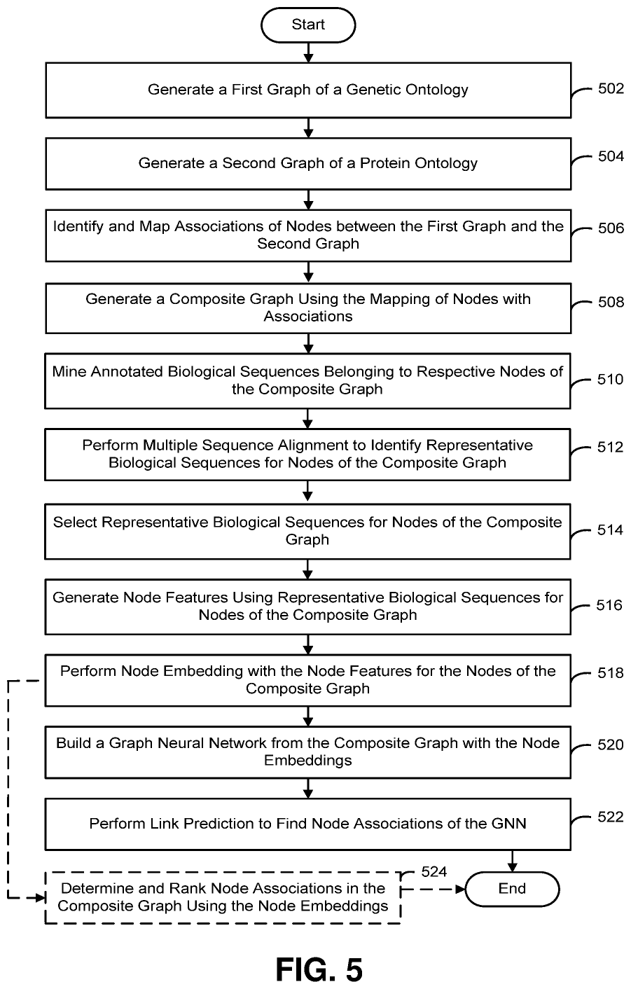

FIGS. 4-5 show flowcharts and/or block diagrams that illustrate the architecture, functionality, and operation of possible implementations of systems, methods, and computer program products according to various embodiments of the present invention. As noted above with respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time. And some blocks shown may be performed and other blocks not performed, depending upon the functionality involved.

FIG. 4 shows a flowchart of an exemplary method in accordance with aspects of the present invention. Steps of the method may be carried out in the environment of FIG. 2 and are described with reference to elements depicted in FIG. 2. In particular, the flowchart of FIG. 4 shows an exemplary method for mapping functional annotations of genes and proteins from different ontologies in accordance with aspects of the present invention.

At step 402, the system generates a first graph of a gene ontology capturing biological processes. For example, a directed acyclic graph G is constructed from the gene ontology information, where G=(V_{G},E_{G}), V_{G} is the set of vertices or nodes representing the terms of the ontology, and E_{G} is the set of edges representing the hierarchical relationship between terms of the ontology. In embodiments, and as described with respect to FIG. 2, graph build module 210 generates the first graph of the gene ontology capturing biological processes.

At step 404, the system generates a second graph of a protein function ontology. For example, directed acyclic graph I is constructed from the protein ontology information, where I=(V_{I},E_{I}), V_{I} is the set of vertices or nodes representing the terms of the ontology, and E_{I} is the set of edges representing the hierarchical relationship between terms of the ontology. In embodiments, and as described with respect to FIG. 2, graph build module 210 generates the second graph of the protein function ontology.

At step 406, the system identifies and maps associations of nodes between the first graph and the second graph. For instance, known relationships between terms of the gene ontology and terms of the protein ontology are identified and retrieved in embodiments from mapping information of the ontologies in term map 236, as described with respect to FIG. 2, and a mapping, M, is created between V_{G} and V_{I} such that the terms of the vertices or nodes of V_{G} from the gene ontology that correspond with the terms of the vertices or nodes of V_{I} from the protein ontology are mapped between graphs G and I. In embodiments, and as described with respect to FIG. 2, term mapping module 212 identifies and maps associations of nodes between the first graph and the second graph.

At step 408, the system generates a composite graph using the mapping of nodes with associations. For example, a composite graph D is generated by merging graphs G and I using mapping M of known associations of nodes between graphs G and I in embodiments. An intermediate graph N may be constructed such that the nodes in N are {V_{G}, V_{I}} and the edges are {E_{G},E_{I}·E_{M}}, where E_{M} are the new edges formed according to the information in mapping. M. The largest connected component (LCC) may be extracted from graph N to form graph D such that D=(V,E) where V and E are the nodes and edges remaining in the LCC of N. In embodiments, and as described with respect to FIG. 2, graph merging module 214 generates a composite graph using the mapping of nodes with associations.

At step 410, the system generates and adds node embeddings to the composite graph. For instance, the node embeddings may be latent multi-dimensional embedding generated in embodiments for each node in D by applying shallow network embedding techniques that preserve the network neighborhood of nodes of the graph, for example, like the DeepWalk technique and node2vec technique that employ a skip-gram model on generated random walks of the graph. In embodiments, and as described with respect to FIG. 2, node embedding module 216 generates and adds node embeddings to the composite graph.

At step 412, the system determines candidate node associations for unassociated nodes of the composite graph using the node embeddings. For example, the proximity of given pairs of node associations (g,i) without a mapping in M, where g∈V_{G} and i∈V_{I}, may be determined in the latent space by exploiting the node embeddings to compute distance measures between the nodes. Two nodes are determined to be close if the distance measure is within a statistical threshold, and, accordingly, are candidates that may be ranked among other candidates for identifying an association between the nodes. In this way, the proximity of the pair of nodes may be determined in the latent space for each node, g, from V_{G} with respect to a given node, i, from V_{I}, where a mapping between given pairs of node associations (g,i) does not exist in M. In embodiments, various strategies can be applied to estimate the statistical threshold by leveraging the information captured through the known relationships in M. For instance, parameters of the distribution generated may be inferred by the distance measures observed between pairs in M and/or by determining the distribution of the length of shortest path between the pairs. A scoring function can be computed by combining certain thresholds to perform a combined ranking. In embodiments, and as described with respect to FIG. 2, node ranking module 218 determines node associations for unassociated nodes of the composite graph using the node embeddings.

At step 414, the system ranks candidate node associations in the composite graph using the node embeddings. In embodiments, and as described with respect to FIG. 2, node ranking module 218 ranks pairs of nodes in the composite graph by distance measures calculated between nodes in the pairs using the node embeddings. Ranked results may be sent in embodiments to user interface 304 for display to user 302, each described with respect to FIG. 2, for queries entered for information about a gene or protein. In embodiments, the highest ranked pair of node associations (g,i) may be saved in persistent storage, for instance in term map 236, described with respect to FIG. 2.

FIG. 5 shows a flowchart of an exemplary method in accordance with aspects of the present invention. Steps of the method may be carried out in the environment of FIG. 2 and are described with reference to elements depicted in FIG. 2. In particular, the flowchart of FIG. 5 shows other exemplary methods for mapping functional annotations of genes and proteins from different ontologies in accordance with aspects of the present invention.

At step 502, the system generates a first graph of a gene ontology capturing biological processes. For example, a directed acyclic graph G is constructed from the gene ontology information, where G=(V_{G},E_{G}), V_{G} is the set of vertices or nodes representing the terms of the ontology, and E_{G} is the set of edges representing the hierarchical relationship between terms of the ontology. In embodiments, and as described with respect to FIG. 2, graph build module 210 generates the first graph of the gene ontology capturing biological processes.

At step 504, the system generates a second graph of a protein function ontology. For example, directed acyclic graph I is constructed from the protein ontology information, where I=(V_{I},E_{I}), V_{I} is the set of vertices or nodes representing the terms of the ontology, and E_{I} is the set of edges representing the hierarchical relationship between terms of the ontology. In embodiments, and as described with respect to FIG. 2, graph build module 210 generates the second graph of the protein function ontology.

At step 506, the system identifies and maps associations of nodes between the first graph and the second graph. For instance, known relationships between terms of the gene ontology and terms of the protein ontology are identified and retrieved in embodiments from mapping information of the ontologies in term map 236, as described with respect to FIG. 2, and a mapping, M, is created between V_{G} and V_{I} such that the terms of the vertices or nodes of V_{G} from the gene ontology that correspond with the terms of the vertices or nodes of V_{I} from the protein ontology are mapped between graphs G and I. In embodiments, and as described with respect to FIG. 2, term mapping module 212 identifies and maps associations of nodes between the first graph and the second graph.

At step 508, the system generates a composite graph using the mapping of nodes with associations. For example, a composite graph D is generated by merging graphs G and I using mapping M in embodiments. An intermediate graph N may be constructed such that the nodes in N are {V_{G}, V_{I}} and the edges are {E_{G}, E_{I}, E_{M}}, where E_{M} are the new edges formed according to the information in mapping, M. The largest connected component (LCC) may be extracted from graph N to form graph D such that D=(V,E) where V and E are the nodes and edges remaining in the LCC of N. In embodiments, and as described with respect to FIG. 2, graph merging module 214 generates a composite graph using the mapping of nodes with associations.

At step 510, the system mines annotated biological sequences to identify DNA sequences belonging to respective nodes of the composite graph. For example, resources such as databases with associations of biological sequences with terms of the gene ontology and/or terms of the protein ontology may be mined to collect DNA sequences associated with the respective terms of the ontologies. An example of a resource with association of biological sequences is the IBM Functional Genomics Platform which is a relational database linking genotype to phenotype for over 300 million biological sequences extracted from microbial genomes. In embodiments, and as described with respect to FIG. 2, node embedding module 216 mines annotated biological sequences to identify DNA sequences belonging to respective nodes of the composite graph.

At step 512, the system performs multiple sequence alignment to identify representative biological sequences for nodes of the composite graph. Identical sequences present in multiple nodes may be removed in embodiments so that representative sequences are unique to their respective nodes. In embodiments, and as described with respect to FIG. 2, MSA module 220 performs multiple sequence alignment to identify representative biological sequences for nodes of the composite graph.

At step 514, the system selects representative biological sequences for nodes of the composite graph. In embodiments, there may be a minimum threshold of one (1) and a predetermined maximum threshold number of representative sequences per node, for example two (2) or three (3). In embodiments, nodes may be removed from the composite graph that have no representative sequence. In embodiments, and as described with respect to FIG. 2, MSA module 220 selects representative biological sequences for nodes of the composite graph.

At step 516, the system generates node features using representative biological sequences for nodes of the composite graph. For instance, latent representations may be generated for each node using DNA sequences as features through standard NLP techniques such as seq2seq and word2vec. For example, seq2seq or a sequence-to-sequence technique encodes each DNA sequence as a latent representation such as a context vector. The latent representations output by the encoder are assigned as features to each node in the composite graph as node attributes. In embodiments, and as described with respect to FIG. 2, feature module 222 generates node features using representative biological sequences for nodes of the composite graph.

At step 518, the system performs node embedding with the node features for the nodes of the composite graph. For example, the node embeddings may include low-dimensional vectors that represent the graph nodes and edges in a vectorial space and context vectors that are latent representations of DNA sequences. In embodiments, and as described with respect to FIG. 2, node embedding module 216 performs node embedding with the node features for the nodes of the composite graph.

At step 520, the system builds a graph neural network from the composite graph with the node embeddings. In embodiments, the composite graph is translated to an adjacency matrix and the node embeddings of features are translated to a node attribute matrix. The adjacency matrix and node attribute matrix are input into stacked layers of the graph neural network that perform computational operations using a convolutional operator, a recurrent operator, a sampling operator, and a skip connection operator to propagate information in each layer and a pooling operator that extract high-level information in each layer. The output layer of the graph neural network generates a matrix of node embeddings with weighted probabilities from the information provided by the stacked layers that may be trained according to a loss function. The trained graph neural network can predict a protein that maps to a given gene and can predict a gene that maps to a given protein. In embodiments, and as described with respect to FIG. 2. GNN build module 224 builds a graph neural network from the composite graph with the node embeddings.

At step 522, the system performs link prediction to find node associations of the graph neural network. In link prediction, the GNN predicts connections between nodes based on node embeddings output by the GNN. The term of the node predicted to be associated with the term of the node input in a query may be sent to user device 240 for display to a user for queries entered for information about a gene or protein. In embodiments, the terms of the nodes predicted to be associated may be saved in term map 236, described with respect to FIG. 2. In embodiments, and as described with respect to FIG. 2, GNN module 226 performs link prediction to find node associations of the graph neural network.

In alternate embodiments of the exemplary method of the steps carried out for mapping functional annotations of genes and proteins from different ontologies, alternate step 524 may be carried out after step 518 instead of carrying out steps 520 and 522.

At step 524, the system determines and ranks node associations in the composite graph using the node embeddings with the node features. For example, the proximity of given pairs of node associations (g,i) without a mapping, where g∈V_{G} and i∈V_{I}, may be determined in the latent space by exploiting the node embeddings to compute distance measures between the nodes. Two nodes are determined to be close if the distance measure is within a statistical threshold, and, accordingly, are candidates that may be ranked among other candidates for identifying an association between the nodes. In this way, the proximity of the pair of nodes may be determined in the latent space for each node, g, from V_{G} with respect to a given node, i, from V_{I}, where a mapping between given pairs of node associations (g,i) is not known. In embodiments, various strategies can be applied to estimate the statistical threshold by leveraging the information captured through the known relationships of node associations. For instance, parameters of the distribution generated may be inferred by the distance measures observed between pairs of known associations and/or by determining the distribution of the length of shortest path between the pairs. A scoring function can be computed by combining certain thresholds to perform a combined ranking. In embodiments, and as described with respect to FIG. 2, node ranking module 218 determines node associations in the composite graph using the node embeddings. In embodiments, and as described with respect to FIG. 2, node ranking module 218 ranks pairs of nodes in the composite graph by distance measures calculated between nodes in the pairs using the node embeddings. Ranked results may be sent in embodiments to user device 240, described with respect to FIG. 2 for display to a user for queries entered for information about a gene or protein. In embodiments, the highest ranked pair of node associations (g,i) may be saved in persistent storage, for instance in term map 236, described with respect to FIG. 2.

In this way, embodiments of the present disclosure map terms and align functional annotations from different ontologies using graph-based artificial intelligence (AI). Advantageously, embodiments of the present disclosure automatically and computationally map terms of gene and protein ontologies and can provide functional annotations available in these ontologies for queries.

In embodiments, a service provider could offer to perform the processes described herein. In this case, the service provider can create, maintain, deploy, support, etc., the computer infrastructure that performs the process steps of the invention for one or more customers. These customers may be, for example, any business that uses technology. In return, the service provider can receive payment from the customer(s) under a subscription and/or fee agreement and/or the service provider can receive payment from the sale of advertising content to one or more third parties.

In still additional embodiments, the invention provides a computer-implemented method, via a network. In this case, a computer infrastructure, such as computer 101 of FIG. 1, can be provided and one or more systems for performing the processes of the invention can be obtained (e.g., created, purchased, used, modified, etc.) and deployed to the computer infrastructure. To this extent, the deployment of a system can comprise one or more of: (1) installing program code on a computing device, such as computer 101 of FIG. 1, from a computer-readable medium; (2) adding one or more computing devices to the computer infrastructure; and (3) incorporating and/or modifying one or more existing systems of the computer infrastructure to enable the computer infrastructure to perform the processes of the invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
identifying, by a processor set, associations of nodes between a first graph of a gene ontology capturing biological processes and a second graph of a protein function ontology;
generating, by the processor set, a composite graph by merging the first graph and the second graph using the associations of nodes;
adding, by the processor set, node embeddings for nodes of the composite graph;
determining, by the processor set, at least one new association of unassociated nodes of the composite graph using the node embeddings; and
saving, by the processor set, the at least one new association of unassociated nodes of the composite graph as an association of nodes between the first graph and the second graph in persistent storage.

2. The method of claim 1, further comprising:
generating the first graph based on information from the gene ontology; and
generating the second graph based on information from the protein ontology.

3. The method of claim 1, further comprising generating the node embeddings for the nodes of the composite graph that preserves a network neighborhood of the nodes of the composite graph.

4. The method of claim 3, wherein the generating comprises applying a shallow network embedding technique that employs a skip-gram model on generated random walks of the composite graph.

5. The method of claim 1, further comprising ranking associations of the at least one new association of unassociated nodes of the composite graph using the node embeddings.

6. The method of claim 5, wherein the ranking comprises determining a distance measure between the nodes of the at least one new association based on the node embeddings.

7. The method of claim 1, wherein the node embeddings comprise latent multi-dimensional embeddings generated for the nodes of the composite graph by applying a shallow network embedding technique that preserves a network neighborhood of the nodes of the composite graph.

8. A computer program product comprising one or more computer readable storage media having program instructions collectively stored on the one or more computer readable storage media, the program instructions executable to:
identify associations of nodes between a first graph of a gene ontology capturing biological processes and a second graph of a protein function ontology;
generate a composite graph by merging the first graph and the second graph using the associations of nodes;
perform node embedding including node features of biological sequences for nodes of the composite graph;
determine at least one new association of unassociated nodes of the composite graph using the node embeddings; and
save the at least one new association of unassociated nodes of the composite graph as an association of nodes between the first graph and the second graph in persistent storage.

9. The computer program product of claim 8, wherein the program instructions are further executable to:
generate the first graph based on information from the gene ontology; and
generate the second graph based on information from the protein ontology.

10. The computer program product of claim 8, wherein the program instructions are further executable to select the biological sequences for the nodes of the composite graph.

11. The computer program product of claim 10, wherein the selecting comprises:
mining annotated sequence ontologies for the biological sequences associated with terms from the nodes of the composite graph; and
performing multiple sequence alignment to identify the biological sequences for the nodes of the composite graph.

12. The computer program product of claim 8, wherein the node embeddings comprise:
low-dimensional vectors that represent the graph nodes and edges in a vectorial space; and
context vectors that are latent representations of DNA sequences.

13. The computer program product of claim 8, wherein the program instructions are further executable to rank associations of the at least one new association of unassociated nodes of the composite graph using the node embeddings.

14. A system comprising:
a processor, a computer readable memory, one or more computer readable storage media, and program instructions collectively stored on the one or more computer readable storage media, the program instructions executable to:
identify associations of nodes between a first graph of a gene ontology capturing biological processes and a second graph of a protein function ontology;
generate a composite graph by merging the first graph and the second graph using the associations of nodes;
perform node embedding for nodes of the composite graph;
build a graph neural network from the composite graph with the node embeddings;
perform link prediction using the graph neural network to identify at least one new association of unassociated nodes of the composite graph; and
save the at least one new association of unassociated nodes of the composite graph as an association of nodes between the first graph and the second graph in persistent storage.

15. The system of claim 14, wherein the program instructions are further executable to:
generate the first graph based on information from the gene ontology; and
generate the second graph based on information from the protein ontology.

16. The system of claim 14, wherein the node embeddings comprise:
low-dimensional vectors that represent the graph nodes and edges in a vectorial space; and
context vectors that are latent representations of DNA sequences.

17. The system of claim 14, wherein the program instructions are further executable to:
obtain annotated biological sequences belonging to nodes of the composite graph;
perform multiple sequence alignment to identify the biological sequences for the nodes of the composite graph; and
select representative biological sequences for nodes of the composite graph.

18. The system of claim 14, wherein the performing comprises:
applying a sequence-to-sequence technique that encodes a DNA sequence as a context vector; and
including the context vector in a node embedding for a node of the composite graph.

19. The system of claim 14, wherein the building comprises:
translating the composite graph to an adjacency matrix;
translating the node embeddings to a node attribute matrix; and
outputting a matrix of probabilities that two nodes of the composite graph are associated.

20. The system of claim 14, wherein the program instructions are further executable to train the graph neural network using the composite graph and the associations of nodes to perform the link prediction.
